(19) United States
(12) Patent Application Publication
(10) Pub. No.: US 2025/0366965 A1
(43) Pub. Date: Dec. 4, 2025

(54) Orthodontic Elastomeric Ligature Ties and Uses Thereof

(71) Applicants: Yan Jing, Dallas, TX (US); Chi Ma, Sunnyvale, TX (US)

(72) Inventors: Yan Jing, Dallas, TX (US); Chi Ma, Sunnyvale, TX (US)

(73) Assignee: The Texas A&M University System, College Station, TX (US)

(21) Appl. No.: 19/223,424

(22) Filed: May 30, 2025

Related U.S. Application Data
(60) Provisional application No. 63/654,466, filed on May 31, 2024.

(51) Int. Cl. A61C 19/06; A61C 7/30; A61K 6/20

(52) U.S. Cl. CPC A61C 19/063; A61C 7/303; A61K 6/20

(57) ABSTRACT

Provided herein is a method for coating an orthodontic elastomeric ligature tie with fluoride in which a solution of a coating medium, for example, polycaprolactone, containing calcium fluoride is coated and dried on the O-ring. Also provided are the coated orthodontic elastomeric ligature ties as prepared by the method and oral devices to re-mineralize decalcified tooth enamel lesions. Methods for treating demineralization in tooth enamel with the coated orthodontic elastomeric ligature ties, for re-mineralizing decalcified tooth enamel lesion in subjects with orthodontia and for optimizing time-release of fluoride from the coated orthodontic elastomeric ligature tie are provided. A kit to prepare the coated orthodontic elastomeric ligature tie is provided.

### CROSS-REFERENCE TO RELATED APPLICATIONS

This non-provisional application claims the benefit of priority under 35 U.S.C. § 119(e) of provisional application U.S. Ser. No. 63/654,466, filed May 31, 2024, the entirety of which is hereby incorporated by reference.

### BACKGROUND OF THE INVENTION

### Field of the Invention

The present invention relates generally to the fields of dentistry and orthodontics. More specifically, the present invention relates to devices and methods to prevent or diminish demineralization of enamel.

### Description of the Related Art

White spot lesions (WSLs) pose substantial esthetic problems for patients (1). Approximately 50% of orthodontic patients develop white spot lesions on at least one tooth (2-5). In some severe cases, the occurrence of white spot lesions during orthodontic treatment may necessitate premature debonding to prevent further damage to the enamel (6-7). Such problems have caused clinicians to search for a solution to prevent orthodontic-associated demineralization.

The incorporation of fluoride into the hydroxyapatite structure of enamel leads to the formation of fluorapatite, which inhibits demineralization due to its lower solubility as well as enhances the remineralization of decalcified enamel lesions by partially reconstructing damaged hydroxyapatite crystal structure (8). When salivary pH drops, the formed hydroxyapatite incorporated into the enamel structure is not a source for fluoride release. Fluoride should be available when the pH value decreases in the oral cavity with proper concentration to increase the ability of the tooth structure to uptake the mineral content needed through the process of remineralization (9).

Fluoride-containing products were developed to increase fluoride uptake by the tooth structures and extend the sustainability of fluoride release. Most fluoride delivery vehicles (fluoride varnish, sealants, and orthodontic adhesives, glass ionomer cement) have a problem with a high release of fluoride in the initial days of its application (burst effect) followed by a rapid drop in fluoride release (5, 10-12). This severely limits the time that the fluoride-releasing materials are effective in preventing white spot lesions (13-21).

The burst effect is caused by the high solubility of the fluoride source, such as tin fluoride or sodium fluoride (19, 20, 22-29). To avoid the burst effect, fluoride needs to be released slowly, as demonstrated with calcium fluoride (CaF₂). The CaF₂ crystals formed on the enamel and within plaque can act as a reservoir, releasing fluoride when the surrounding environment shifts toward the acidic side, leading to decreased demineralization and increased remineralization (30-33). The low solubility of CaF₂ allows the fluoride ions to be slowly released over a longer period of time. However, the formation of CaF₂ during dental care is limited due to the low availability of calcium ions in saliva (30). This problem can be circumvented by the addition of exogenous calcium fluoride particles to dental care products.

O-rings are small rubber elastomeric ligature to support the arch-wire's attachment to each bracket. The integration of fluoride into elastomeric rings for the prevention of white spot lesions in orthodontic patients has been explored (19, 20, 22-29). Those products have been discontinued, however, due to the severe initial burst effect of fluoride release, the swelling and imbibition in oral application, and limited effects on white spot lesion prevention.

Polycaprolactone (PCL) is approved for use in surgical implants and drug delivery devices for tissue engineering and regenerative medicine applications due to its biosafety. Polycaprolactone is partially crystalline and has a low glass transition temperature of −60° C. This allows polycaprolactone and its composites an extraordinary tensile extensibility (37).

Thus, there is a need in the art for devices and methods to prevent or diminish demineralization of enamel during and after fixed orthodontic treatment so as to strengthen the enamel and reduce the occurrence of white spot lesions. The present invention fulfills this long-standing need in the art.

### SUMMARY OF THE INVENTION

The present invention is directed to a method for coating an orthodontic elastomeric ligature tie (O-ring) with fluoride. In this method, a solution of a coating medium and calcium fluoride microcrystals is prepared and coating the O-ring is coated with the solution. The solution is dried on the O-ring to form a layer of the coating medium with the calcium fluoride microcrystals embedded therein. The present invention is directed to a related method further comprising increasing a concentration of the coating medium during the preparing step such that a thicker layer with an increase in an availability of fluoride therein is formed on the O-ring during the coating step.

The present invention is further directed to an orthodontic elastomeric ligature tie (O-ring) prepared by the method as described herein.

The present invention is directed further to a method for treating demineralization of tooth enamel during an orthodontic treatment in a subject in need thereof. In this method, the orthodontic elastomeric ligature tie (O-ring) as described herein is attached to braces on the teeth, where a concentration of the coating medium extends a time-release of the fluoride embedded therein as calcium fluoride. The fluoride is incorporated into the enamel thereby decreasing or preventing demineralization in the tooth enamel.

The present invention is directed further still to a method for optimizing time-release of fluoride coated onto an orthodontic elastomeric ligature tie (O-ring). In this method, the O-ring is coated with a polycaprolactone coating medium containing calcium fluoride microcrystals embedded within a matrix thereof, where an increase in optimization of the time-release of the fluoride from the polycaprolactone matrix coating the O-ring correlates to a concentration of the polycaprolactone thereon.

The present invention is directed further still to an oral device to re-mineralize decalcified tooth enamel lesions. The oral device has an orthodontic elastomeric ligature tie (O-ring) and a coating medium that comprises a polycaprolactone matrix disposed thereon and containing calcium fluoride microcrystals embedded therein.

The present invention is directed further still to a method for re-mineralizing decalcified tooth enamel lesions in a subject with orthodontia. In this method, securing the oral device described herein is secured to the orthodontia in the mouth of the subject. The fluoride from the calcium fluoride embedded within the polycaprolactone matrix is time-released which is dependent on the concentration of the polycaprolactone within the polycaprolactone, where the concentration is about 2.5% to about 10%.

The present invention is directed further still to a kit for modifying an orthodontic elastomeric ligature tie (O-ring) to release fluoride as a therapeutic. The kit comprises polycaprolactone, calcium fluoride, at least one solvent to prepare a coating medium of the polycaprolactone and calcium fluoride, and instructions to prepare calcium fluoride coated O-rings.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1F show that the polycaprolactone layer incorporated with calcium fluoride was successfully coated on the surface of the elastomeric ligature tie. FIG. 1A: The white polycaprolactone layer shown on 2.5%, 5% and 10% groups of Ca—F elastomeric ligature ties. FIG. 1B: low magnification SEM images for the cross sections of the four groups of Elastomeric ligature ties. FIG. 1C: high magnification SEM images for the boundary between the polycaprolactone layer and the Elastomeric ligature tie matrix (red double-arrows representing the PCL layers). FIG. 1D: the quantitation data of the thickness of the polycaprolactone layer in the three groups of Ca—F Elastomeric ligature ties. FIG. 1E: the absorbance peaks of fluoride and calcium ions from energy-dispersive x-ray spectroscopy analysis shown in the polycaprolactone matrix but not in the Elastomeric ligature tie matrix. FIG. 1F: quantitation results of the EDX measurements for the element mass percentage of fluoride and calcium in the three groups of Ca—F elastomeric ligature ties. **P<0.01; ***P<0.001.

FIGS. 2A-2C show the fluoride releasing profile of the Ca—F Elastomeric ligature ties. FIG. 2A: The fluoride release rate in the first week. FIG. 2B: the weekly fluoride release rate. FIG. 2C: the cumulative fluoride release over 7 weeks.

FIGS. 3A-3C shows the elastic property of Ca—F Elastomeric ligature ties. FIG. 3A: The Instron test results after elastomeric ligature tie modification (T1). FIG. 3B: the Instron test results after 7-week release (T2). FIG. 3C: the difference in Instron test results between T1 and T2 in the four groups of Elastomeric ligature ties.

FIGS. 4A-4C show the elastic curves of the four groups of Elastomeric ligature ties from the Instron mechanical tests. FIG. 4A: The curves after Elastomeric ligature tie modification (T1). FIG. 4B: the curves after 7-week release (T2). FIG. 4C: the appearance of the control and Ca—F Elastomeric ligature ties after 7-week soaking.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention.

As used herein, the term “or” in the claims refers to “and/or” unless explicitly indicated to refer to alternatives only or the alternatives are mutually exclusive, although the disclosure supports a definition that refers to only alternatives and “and/or”.

As used herein, the terms “comprise” and “comprising” are used in the inclusive, open sense, meaning that additional elements may be included. Correspondingly, the terms “consists of” and “consisting of” are used in the exclusive, closed sense, meaning that additional elements may not be included.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

In one embodiment of the present invention, there is provided a method for coating an orthodontic elastomeric ligature tie (O-ring) with fluoride, comprising preparing a solution of a coating medium and calcium fluoride microcrystals; coating the O-ring with the solution; and drying the solution on the O-ring to form a layer of the coating medium with the calcium fluoride microcrystals embedded therein. Further to this embodiment, the method comprises increasing a concentration of the coating medium during the preparing step such that a thicker layer with an increase in an availability of fluoride therein is formed on the O-ring during the coating step.

In both embodiments, the coating medium may be polycaprolactone (PCL). Particularly, the polycaprolactone may be at a concentration of about 2.5% to about 10% in the solution. Preferably, the polycaprolactone is at a concentration of 5% in the solution or 10% in the solution.

In another embodiment of the present invention, there is provided an orthodontic elastomeric ligature tie (O-ring) prepared by the method as described supra. In this embodiment, the O-ring may comprise a coating of about 5% to about 10% polycaprolactone with calcium fluoride microcrystals embedded therein.

In yet another embodiment of the present invention, there is provided a method for treating demineralization of tooth enamel during an orthodontic treatment in a subject in need thereof, comprising attaching the orthodontic elastomeric ligature tie (O-ring) as described supra to braces on the teeth, a concentration of the coating medium extending time-release of the fluoride embedded therein as calcium fluoride; and incorporating the fluoride into the enamel thereby decreasing or preventing demineralization in the tooth enamel.

In this embodiment, incorporating the fluoride into the enamel may further increase remineralization of decalcified tooth enamel lesions. In this embodiment, the coating medium may be polycaprolactone, where an increasing concentration thereof on the O-ring increases availability of the fluoride therein to the tooth enamel. Particularly, the concentration of polycaprolactone may be about 2.5% to about 10%. Preferably, the concentration of polycaprolactone is 5% or 10%.

In yet another embodiment of the present invention, there is provided a method for optimizing time-release of fluoride coated onto an orthodontic elastomeric ligature tie (O-ring); comprising coating the O-ring with a polycaprolactone coating medium containing calcium fluoride microcrystals embedded within a matrix thereof, where an increase in optimization of the time-release of the fluoride from the polycaprolactone matrix coating the O-ring correlates to a concentration of the polycaprolactone thereon. In this embodiment, the concentration of polycaprolactone may be about 5% to about 10%.

In yet another embodiment of the present invention, there is provided an oral device to re-mineralize decalcified tooth enamel lesions, comprising an orthodontic elastomeric ligature tie (O-ring); and a coating medium comprising a polycaprolactone matrix disposed thereon and containing calcium fluoride microcrystals embedded therein.

In this embodiment, the polycaprolactone may have a concentration of about 2.5% to about 10% within the matrix. Particularly, the polycaprolactone has a concentration of about 5% or about 10% within the matrix. Further in this embodiment, a time-release of fluoride from the polycaprolactone matrix may correlate to the concentration of polycaprolactone therein.

In yet another embodiment of the present invention, there is provided a method for re-mineralizing decalcified tooth enamel lesions in a subject with orthodontia, comprising securing the oral device as described supra to the orthodontia in the mouth of the subject; and time-releasing fluoride from the calcium fluoride embedded within the polycaprolactone matrix, where the time-releasing is dependent on the concentration of the polycaprolactone within the polycaprolactone matrix; where the concentration is about 2.5% to about 10%.

In yet another embodiment of the present invention, there is provided a kit for modifying an orthodontic elastomeric ligature tie (O-ring) to release fluoride as a therapeutic, comprising polycaprolactone; calcium fluoride; at least one solvent to prepare a coating medium of the polycaprolactone and calcium fluoride; and instructions to prepare calcium fluoride coated O-rings.

The following examples are given for the purpose of illustrating various embodiments of the invention and are not meant to limit the present invention in any fashion.

### Example 1

### Materials and Methods

### Preparation of Fluoridated O-Rings

The Ca—F-coated O-rings were prepared by a simple dip and dry method. First, the Ca—F coating medium was prepared by mixing solutions A and B with 1 to 1 (v/v). Specifically, solution A was prepared by dispersing CaF₂ micro crystals in acetone (400 mg/ml). Three different concentrations of Solution B were prepared by dissolving 5%, 10%, or 20% of PCL (Sigma, 440744) in acetone. 0.5 ml of Solution A and B were mixed to obtain three Ca—F coating media: 2.5%, 5% or 10% PCL with CaF₂. Second, the ordinary O-rings were incubated into a coating medium for one second and transferred into a dry oven for slow solvent evaporation for 1 hour. After that, all the coated O-rings were moved into a vacuum chamber for additional drying for 24 hours. A total of four groups of O-rings were set: 1) ordinary O-rings (control group); 2) Ca—F O-rings coated with 2.5% PCL (2.5% group); 3) Ca—F O-rings coated with 5% PCL (5% group); and 4) Ca—F O-rings coated with 10% PCL (10% group).

Pilot studies have been completed to determine the basis of the dip concentrations and the necessary effect size. With the proposed sample size, 80% power can be attained with an effect size of 2.5. Each group had 32 samples randomly allocated to Scanning Electron Microscopy SEM (n=8), Instron (n=8), and fluoride release profiles (n=16) to analyze the amount of coated calcium fluoride, the elastic properties, and the fluoride release amounts, respectively.

### Scanning Electron Microscopy

Scanning electron microscopy (SEM) was used to characterize the morphology of the O-rings and measure the cross-sectional thickness of the PCL layer after O-ring preparation (T1). The Ca—F O-rings were rinsed in liquid nitrogen for 3 minutes, and then snapped into pieces to expose the cross-sectional surface. The surface was coated with gold for SEM scanning (39, 40). To determine the amount of fluoride and calcium ions in the polycaprolactone layers, energy-dispersive x-ray spectroscopy (EDX) associated with SEM (EDX/SEM) mapping was used to detect the absorbance peaks of fluoride and calcium ions, and to quantify the mass ratio of calcium and fluoride in each group as described (40). This provided the means to determine if calcium and fluoride ions were successfully incorporated into the polycaprolactone layer on the O-ring surface. The cross-section of each O-ring was divided into four quarters, and three points were randomly selected from each quarter. The average of a total of 12 points was used to represent the thickness of the polycaprolactone layer and the EDX results for each O-ring.

### In Vitro Test of Fluoride Release

All O-rings were placed on brackets for the maxillary lateral incisor (American Orthodontics, WI) to mimic the clinical application (41). Every four O-rings were immersed in 10 ml of incubation medium (distilled water) in a 50 ml centrifuge tube. Each group had 4 tubes with a total of 16 O-rings per group. All tubes were incubated at 37° C. during the releasing period. An Ion Selective Electrode (ISE) Meter (Thermo Scientific Orion Dual Star) was used to determine the amount of the released fluoride, and the measurements were taken for 10 time points in total, including Day 1, Day 3, Day 5, Week 1, Week 2, Week 3, Week 4, Week 5, Week 6, and Week 7. The ISE meter was calibrated at each time point of measurement, following the manufacturer's instructions. After the measurement at each time, the O-rings were transferred to new tubes with 10 ml of new incubation medium until the last measurement.

### Mechanical Test

The machinal performance of the Ca—F O-rings was evaluated with Instron (Instron® 5567). The tests were taken at two-time points: before soaking in distilled water (T1), and after soaking in distilled water for 7 weeks (T2). The testing parameters were programmed according to ISO 21606:2007 (42). Briefly, the specimen was stretched at a rate of 100 mm/min to the amplitude of four times the outer diameter (OD) of the O-ring and held for 5 seconds. Then, the O-ring was returned to the amplitude of three times OD at the rate of 100 mm/min. Finally, the specimen was held for 30 seconds, and the tensile force was recorded. If the O-rings were unable to be stretched to the four times folds, then tensile failure was noted. Maximum force (N) at the time of breakage was recorded. If no breakage occurred, the final force (N) of the O-ring was recorded at the end of the test.

### Statistical Analysis

Independent t-tests were used to compare groups for SEM, fluoride release and Instron experiments. ANOVA was used to compare between more than two groups based on dip concentration. The preparation of O-rings, fluoride release testing, and Instron mechanical testing were all carried out by one principal investigator. Reliability was established for fluoride release testing by each fluoride measurement with the ISE Meter repeated 3 times. Intraclass correlation coefficient (ICC) was established at 99.9%.

### Results

### Surface Morphological Structure of Ca—F O-Ring

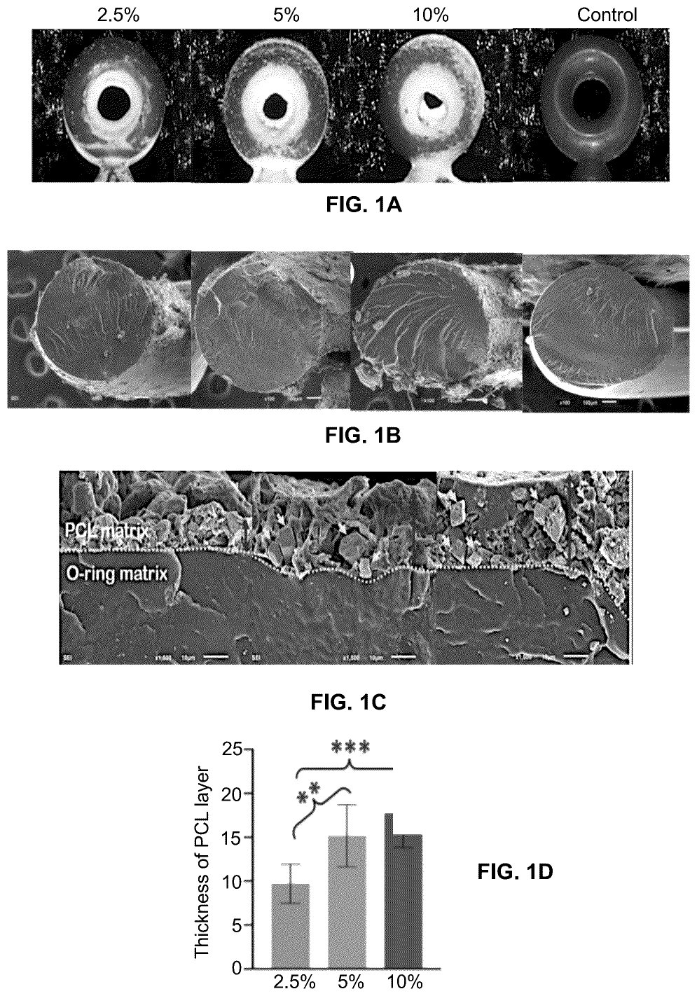

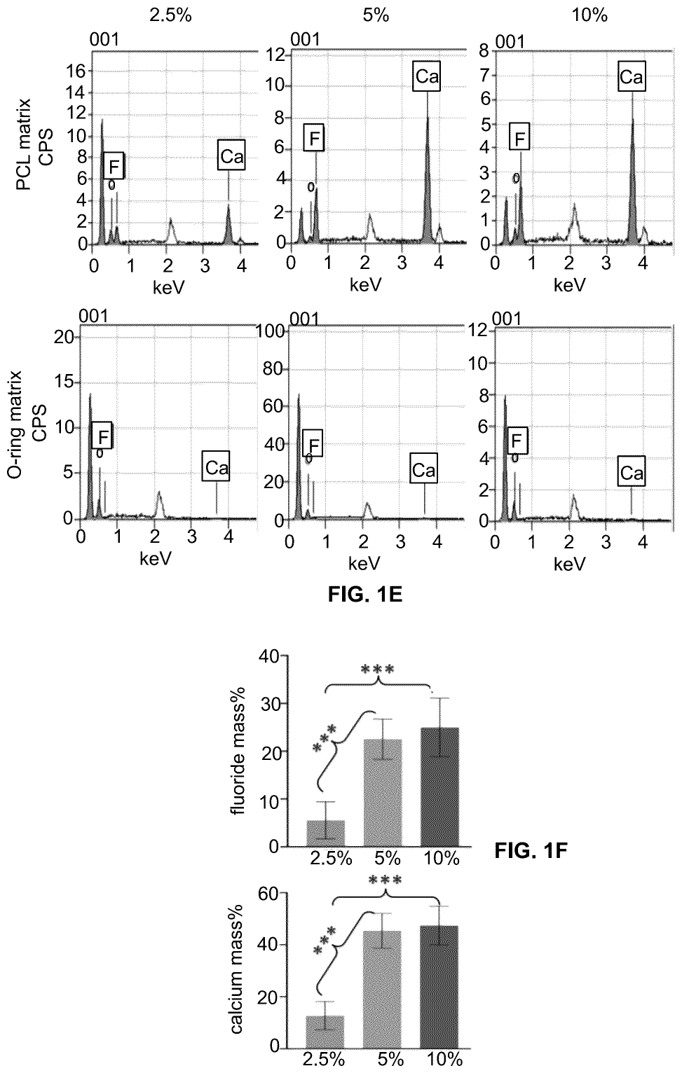

The three sets of Ca—F O-rings exhibited a distinct white polycaprolactone layer on their surfaces (FIG. 1A). At lower magnification (T1), SEM images displayed a coated polycaprolactone layer atop the Ca—F O-ring matrix, while the surface of regular O-rings remained smooth and retained their original appearance (FIG. 1B). SEM images in higher magnification (FIG. 1C) revealed a gradual increase in polycaprolactone layer thickness as the concentration elevated from 2.5% (9.70 μm±2.25 μm) to 5% (15.15 μm≅3.55 μm) and 10% (17.73 μm±3.91 μm), a finding corroborated by quantitative data (FIG. 1D). The difference in the thickness is related to the polycaprolactone solution viscosity. As the increase of the polycaprolactone components, more CaF₂ crystals were embedded within the polycaprolactone matrix (FIG. 1C, yellow arrows).

Energy-dispersive x-ray spectroscopy analyses exhibited absorbance peaks for fluoride and calcium ions within the polycaprolactone matrix of the 2.5%, 5%, and 10% groups, whereas no fluoride or calcium ions were detected in the control O-ring matrix (FIG. 1E). Quantitative analysis results revealed that the element mass percentage of fluoride was significantly increased with the increased concentrations of polycaprolactone (2.5% group, 5.55%±3.89%; 5% group, 22.49%±4.23%; 10% group, 24.97%±6.15%; FIG. 1F, upper panel). The element mass percentage of calcium followed a similar pattern (2.5% group, 12.70%±5.40%; 5% group, 45.36%±6.70%; 10% groups, 47.35%±7.45%; FIG. 1F, lower panel).

These data demonstrated the successful integration of calcium fluoride into the polycaprolactone layer on the O-rings. The thickness of the polycaprolactone layer and the amount of fluoride ion in the polycaprolactone was increased along with the increase of polycaprolactone concentration.

### In Vitro Fluoride Release of Ca—F O-Rings

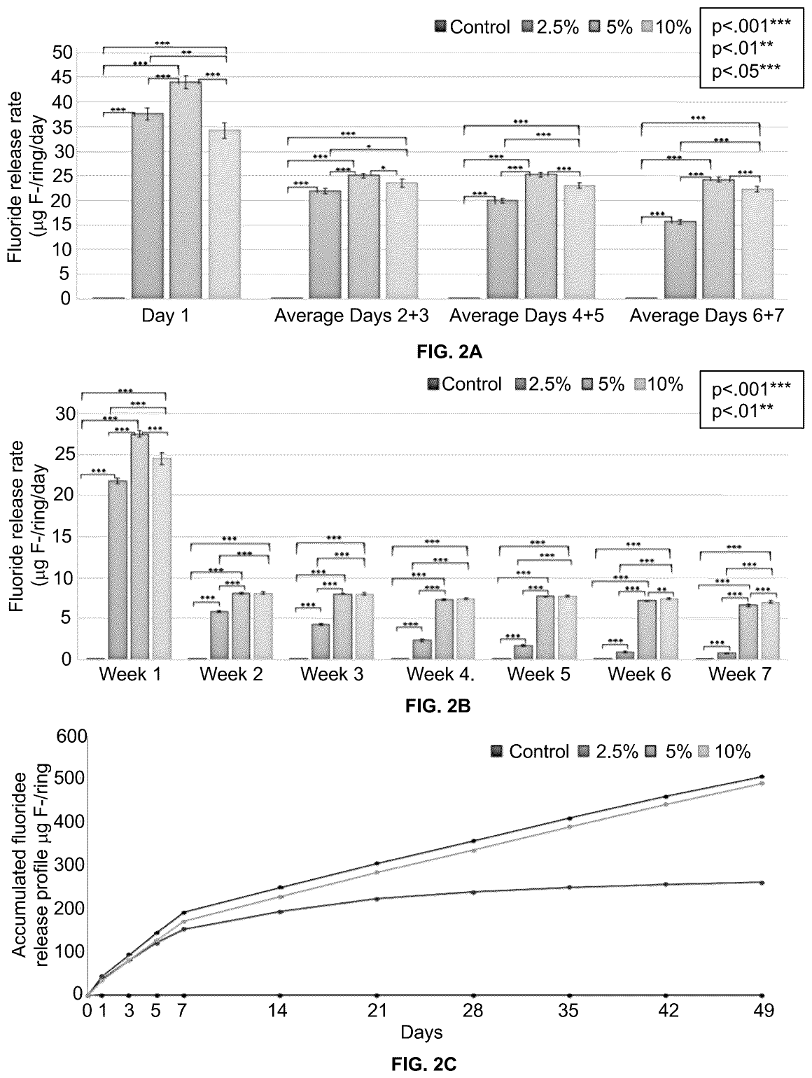

In the initial week, the fluoride burst effect was examined by analyzing the release rates on Day 1 and averaging the rates of Days 2-3, Days 4-5, and Days 6-7. The rate in all experimental groups exhibited a significant decrease from Day 1 to Days 2-3. It subsequently persisted in the 5% and 10% groups throughout the week but not in 2.5% group (Table 1a, FIG. 2A). Fluoride release in the control group was minimal, and there were significant differences between the groups in the first week (Table 1b, FIG. 2A).

TABLE 1A
Daily Fluoride Release Rate in the 1^{st} Week (μg F⁻/ring/day)

| | Control | | | 2.5% | | | 5% | | | 10% | | |
|---|---|---|---|---|---|---|---|---|---|---|---|---|
| | | Std. | | | Std. | P- | | Std. | P- | | Std. | P- |
| | Mean | Dev. | P-value | Mean | Dev. | value | Mean | Dev. | value | Mean | Dev. | value |
| Day 1 | .170 | .028 | .005 | 37.521 | 1.220 | <.001 | 43.958 | 1.228 | <.001 | 34.146 | 1.577 | <.001 |
| μg F⁻/ring/day | | | | | | | | | | | | |
| Day 2 + 3 | 0.35 | .022 | .120 | 21.896 | .580 | .026 | 25.000 | .458 | .026 | 23.458 | .817 | .120 |
| 2 | | | | | | | | | | | | |
| μg F⁻/ring/day | | | | | | | | | | | | |
| Day 4 + 5 | .019 | .008 | | 19.969 | .472 | <.001 | 25.177 | .512 | .025 | 22.969 | .515 | .001 |
| 2 | | | | | | | | | | | | |
| μg F⁻/ring/day | | | | | | | | | | | | |
| Day 6 + 7 | .025 | .009 | .270 | 15.646 | .498 | | 24.135 | .485 | | 22.404 | .504 | |
| 2 | | | | | | | | | | | | |
| μg F⁻/ring/day | | | | | | | | | | | | |

TABLE 1B
Daily Fluoride Release Rate in the 1^{st} Week (μg F⁻/ring/day)

| | Control | | 2.5% | | 5% | | 10% | |
|---|---|---|---|---|---|---|---|---|
| | Mean | Std. Dev. | Mean | Std. Dev. | Mean | Std. Dev. | Mean | Std. Dev. |
| Day 1 | .170 | .028 | 37.521 | 1.220 | 43.958 | 1.226 | 35.146 | 1.577 |
| μg F⁻/ring/day | | | | | | | | |
| Day 2 + 3 | .035 | .022 | 21/896 | .580 | 25.000 | .458 | 23.458 | .817 |
| 2 | | | | | | | | |
| μg F⁻/ring/day | | | | | | | | |
| Day 4 + 5 | .019 | .008 | 10.060 | .472 | 25.177 | .512 | 22.069 | .515 |
| 2 | | | | | | | | |
| μg F⁻/ring/day | | | | | | | | |
| Day 6 + 7 | .025 | .009 | 15.546 | .408 | 24.135 | .485 | 22.240 | .504 |
| 2 | | | | | | | | |
| μg F⁻/ring/day | | | | | | | | |

| | 2.5% vs. Control | 5% vs. Control | 10% vs. Control | 2.5% vs. 5% | 2.5% vs. 10% | 5% vs. 10% |
|---|---|---|---|---|---|---|
| | P-value | P-value | P-value | P-value | P-value | P-value |
| Day 1 | <.001 | <.001 | <.001 | <.001 | <.001 | < 001 |
| μg F⁻/ring/day | | | | | | |
| Day 2 + 3 | <.001 | <.001 | <.001 | <.001 | .001 | .001 |
| 2 | | | | | | |
| μg F⁻/ring/day | | | | | | |
| Day 4 + 5 | <.001 | <.001 | <.001 | <.001 | <.001 | <.001 |
| 2 | | | | | | |
| μg F⁻/ring/day | | | | | | |
| Day 6 + 7 | <.001 | <.001 | <.001 | <.001 | <.001 | <.001 |
| 2 | | | | | | |
| μg F⁻/ring/day | | | | | | |

The long-term release profile was then assessed. The daily release rates from week 1 to week 7 were calculated by averaging the weekly release. All groups experienced a significant decline in fluoride release during the second week compared to the first week (Table 2a), and the release remained consistent for the subsequent time points in 5% and 10% groups but not 2.5% group (Table 2b, FIG. 2B). By the final 7^{th} week, the average fluoride release rates were 0.69 μg F⁻/ring/day, 6.54 μg F⁻/ring/day, and 6.97 μg F⁻/ring/day for the 2.5%, 5%, and 10% groups, respectively.

TABLE 2A
Daily Fluoride Release Rate by Week (μg F⁻/ring/day)

| | Control | | | 2.5% | | | 5% | | | 10% | | |
|---|---|---|---|---|---|---|---|---|---|---|---|---|
| | | Std. | | | Std. | P. | | Std. | P- | | Std | P- |
| | Mean | Dev. | P-value | Mean | Dev | value | Mean | Dev. | value | Mean | Dev. | value |
| Week 1 | .046 | .001 | .019 | 21.792 | .334 | <.001 | 27.512 | .384 | <.001 | 24.497 | .704 | <.001 |
| μg F⁻/ring/day | | | | | | | | | | | | |
| Week 2 | .007 | .002 | | 5.795 | .144 | | 8.042 | .139 | | 8.03 | .157 | |
| μg F⁻/ring/day | | | | | | | | | | | | |
| Week 3 | .005 | .001 | .044 | 4.211 | .106 | <.001 | 7.976 | .063 | .256 | 7.982 | .132 | .320 |
| μg F⁻/ring/day | | | | | | | | | | | | |
| Week 4 | .005 | .001 | .232 | 2.266 | .129 | <.001 | 7.280 | .136 | .001 | 7.254 | .114 | <.001 |
| μg F⁻/ring/day | | | | | | | | | | | | |
| Week 5 | .005 | .001 | .370 | 1.607 | .126 | .001 | 7.682 | .074 | .001 | 7.714 | .129 | .001 |
| μg F⁻/ring/day | | | | | | | | | | | | |
| Week 6 | .005 | .001 | .151 | .895 | .130 | <.001 | 7.098 | .096 | <.001 | 7.342 | .086 | .002 |
| μg F⁻/ring/day | | | | | | | | | | | | |
| Week 7 | .005 | .001 | .436 | .686 | .016 | .037 | 6.535 | .145 | <.001 | 6.970 | .140 | .003 |
| μg F⁻/ring/day | | | | | | | | | | | | |

TABLE 2B
Daily Fluoride Release Rate by Week (μg F⁻/ring/day)

| | Control | | 2.5% | | 5% | | 10% | |
|---|---|---|---|---|---|---|---|---|
| | Mean | Std. Dev. | Mean | Std. Dev. | Mean | Std. Dev. | Mean | Std. Dev. |
| Week 1 | .046 | .011 | 21.792 | .334 | 27.512 | .384 | 24.497 | .704 |
| μg F⁻/ring/day | | | | | | | | |
| Week 2 | .006 | .992 | 5.795 | .144 | 8.042 | .139 | 8.063 | .157 |
| μg F⁻/ring/day | | | | | | | | |
| Week 3 | .995 | .001 | 4.211 | .106 | 7.076 | .063 | 7.082 | .132 |
| μg F⁻/ring/day | | | | | | | | |
| Week 4 | .005 | .001 | 2.266 | .129 | 7.280 | .136 | 7.354 | .114 |
| pg F⁻/ring/day | | | | | | | | |
| Week 5 | .005 | .001 | 1.697 | .126 | 7.682 | .074 | 7.714 | .129 |
| μg F⁻/ring/day | | | | | | | | |
| Week 6 | .005 | .001 | .895 | .110 | 7.098 | .096 | 7.342 | .086 |
| μg F⁻/ring/day | | | | | | | | |
| Week 7 | .005 | .001 | .686 | .16 | 6.539 | .145 | 6.970 | .140 |
| μg F⁻/ring/day | | | | | | | | |

| | 2.5% vs. Control | 5% vs. Control | 10% vs. Control | 2.5% vs. 5% | 2.5% vs. 10% | 5% vs. 10% |
|---|---|---|---|---|---|---|
| | P-value | P-value | P-value | P-value | P-value | P-value |
| Week 1 | <.001 | <.001 | <.001 | <.001 | <.001 | <.001 |
| μg F⁻/ring/day | | | | | | |
| Week 2 | <.001 | <.001 | <.001 | <.001 | <.001 | 1.000 |
| μg F⁻/ring/day | | | | | | |
| Week 3 | <.001 | <.001 | <.001 | <.001 | <.001 | 1.000 |
| μg F⁻/ring/day | | | | | | |
| Week 4 | <.001 | <.001 | <.001 | <.001 | <.001 | 1.000 |
| μg F⁻/ring/day | | | | | | |
| Week 5 | <.001 | <.001 | <.001 | <.001 | <.001 | 1.000 |
| μg F⁻/ring/day | | | | | | |
| Week 6 | <.001 | <.001 | <.001 | <.001 | <.001 | .009 |
| μg F⁻/ring/day | | | | | | |
| Week 7 | <.001 | <.001 | <.001 | <.001 | <.001 | <.001 |
| μg F⁻/ring/day | | | | | | |

The cumulative release curve illustrates fluoride release throughout the 7-week testing period. A linear fluoride release was observed in the groups of 5% and 10%, whereas the release rate reached a plateau after 3 weeks in the 2.5% group (FIG. 2C). The total amount of released fluoride from each ring in 2.5%, 5% and 10% groups are 260.8 μg. 504.9 μg and 489.5 μg, respectively.

Taken together, Ca—F coating sustains the fluoride with low-level burst release. The 5% and 10% groups of Ca—F O-rings exhibited long-lasting fluoride release with a range of 6.54-45 μg F⁻/ring/day in a seven-week experimental period.

### Mechanical Performance of Ca—F O-Rings

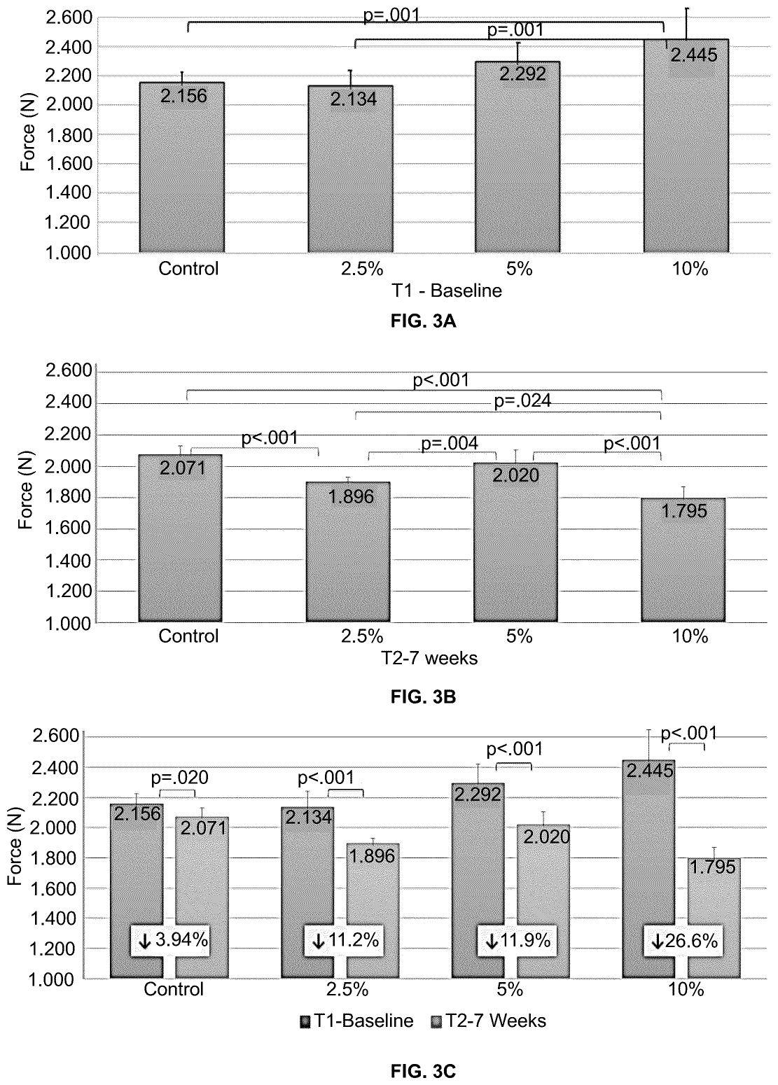

Tensile failure was not observed in any group at any time point. At T1, the tensile force in the 2.5% group (2.13±0.11 N) slightly decreased compared to controls (2.16±0.07 N), while the 5% (2.29±0.13 N) and 10% (2.45±0.20 N) groups showed a moderate and significant increase compared to controls, respectively (FIG. 3A). By T2, the tensile forces of 2.5% (1.90±0.03 N), 5% (2.02±0.08 N) and 10% (1.79±0.07 N) groups all decreased compared to controls (2.07±0.06 N), with statistically significant reductions noted in the 2.5% and 10% groups (FIG. 3B).

Comparing T1 to T2, both control and Ca—F O-rings displayed statistically significant decreases in tensile force (FIG. 3C). Within the groups, percentage decreases in tensile force were −3.94% for controls, −11.2% for the 2.5% group, −11.9% for the 5% group, and −26.6% for the 10% group.

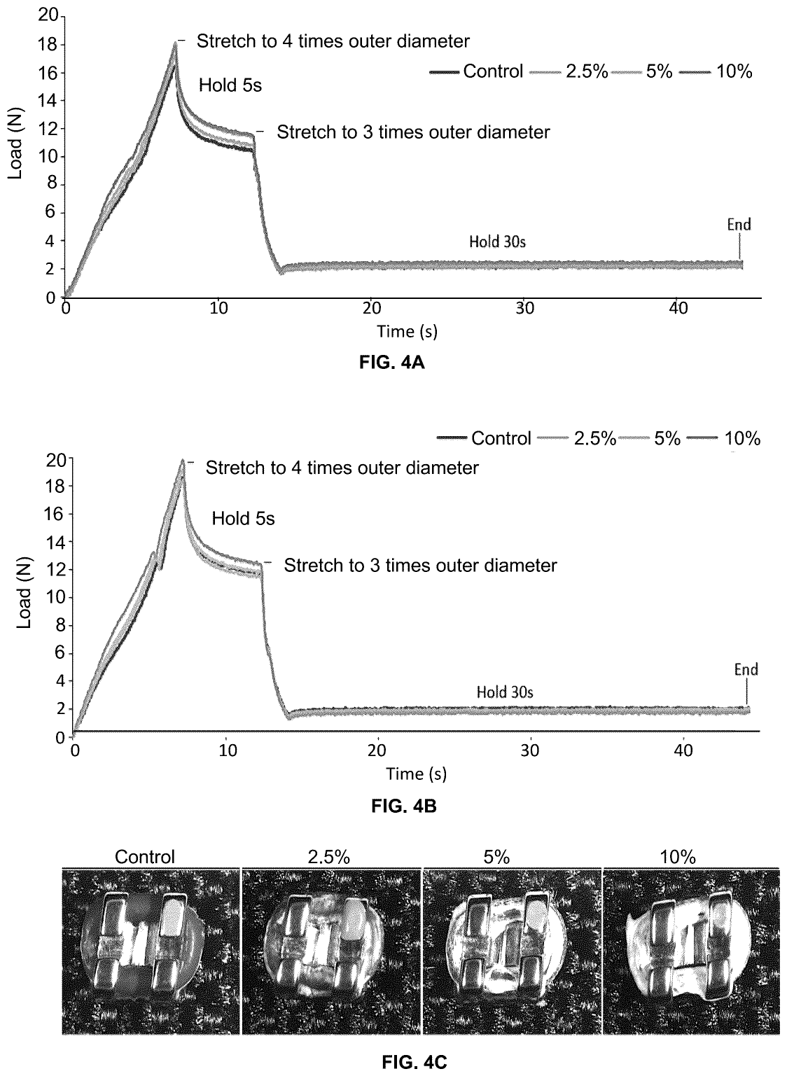

Throughout the mechanical test, all groups exhibited similar elastic curves at both T1 (FIG. 4A) and T2 (FIG. 4B). Additionally, all Ca—F O-rings maintained their structural integrity on the brackets at T2 (FIG. 4C). Taken together, the Ca—F O-rings maintained their elastic properties.

### Discussion

The present invention demonstrates the Ca—F O-rings exhibited long-lasting fluoride release with a therapeutic dose. During the first week, the fluoride release rates of the 2.5%, 5%, and 10% groups exceeded the therapeutic range (1.2-2.8 μg F⁻/ring/day) but remained below the toxic level (51 μg F⁻/ring/day). From the 2nd to the 7th week, the 5% and 10% groups, the release remained linear. In the final week, the fluoride release rates of the 5% and 10% groups still exceeded the therapeutic range, while the 2.5% group fell below the therapeutic range by the 6th week. Collectively, the 5% and 10% groups demonstrated long-term sustained fluoride release within the therapeutic range for at least 7 weeks, meeting the common requirement of orthodontic follow-up intervals (4-6 weeks).

The relatively high burst release (in percentage) and reduction in fluoride release rate in the 2.5% group compared to the 5% and 10% groups could be attributed to the thinner polycaprolactone layer, leading to less fluoride incorporation during the coating (FIG. 1B). The thickness of polycaprolactone layers in 5% and 10% groups are 1.8- and 1.5-fold of that in 2.5% groups, respectively, while the total amount of released fluoride from each ring in the 5% and 10% groups are 1.9- and 1.8-fold of that in the 2.5% group, which are correlated with the differences in the thickness of the coating. In the 10% group, a thicker and smoother polycaprolactone layer was observed, which embedded the CaF2 crystals, producing a more sustained fluoride release than the other two groups.

The fluoride release profiles of the 5% and 10% groups showed no statistically significant differences. This could be due to a limit on the amount of fluoride that can be released from the polycaprolactone medium surrounding the O-rings. Another possibility is that there is a maximum amount of polycaprolactone required for effective, sustained fluoride release, beyond which additional polycaprolactone does not result in a significant difference. Therefore, if assuming 5% as the maximum amount of polycaprolactone that can be loaded into the outer layer of the O-ring for fluoride release, further increasing the concentration, as in the 10% group, might not enhance fluoride release. It is plausible that the 5% group has reached a plateau in the efficacy of PCL, while the 10% group does not provide additional benefits for fluoride release.

The present invention demonstrated significant improvements in sustained fluoride release and burst effect control. An in vitro study using stretched Fluor-I-Tie O-rings in distilled water revealed an initial release of 9.68 μg F⁻/ring/day on the first day. However, by the 7th day, this fluoride release decreased significantly to 0.79 μg F⁻/ring/day, and the overall average release rate over 196 days was 0.71 μg F⁻/ring/day, falling well below the therapeutic range (41). Another in vitro study using Fluor-I-Ties found that 35% of the total fluoride content was released within the first 24 hours, followed by 63% released by the end of the first week (19). In a previous study utilizing a polymer medium and sodium fluoride, fluoride release rates reached 115 μg F⁻/ring on the first day, far exceeding the toxic limit of 51 μg F⁻/ring/day (6). After this initial burst, the fluoride release rate averaged around 6.7 μg F⁻/ring/day for the first 10 days, later dropping to 0.88 μg F⁻/ring/day for the remaining 40 days, falling below the therapeutic range.

In the present study, the burst release (the release in the first week) was more pronounced in the 2.5% group, with 14% released on the first day and 74% released by the end of the first week. In contrast, the 5% and 10% groups demonstrated significant improvements in resolving the burst effect issue. The 5% group released 8.7% of the total fluoride on the first day, followed by 38% by the end of the first week, while the 10% group released 6.9% of total fluoride on the first day, followed by 35% released by the end of the first week. Both the 5% and 10% groups maintained linear and therapeutic release levels from the 2^{nd} to the 7^{th} week, showcasing substantial progress in sustained fluoride release and mitigating the burst effect.

The present invention demonstrated an improvement in sustained fluoride release and the reduction of the burst effect and is the first instance of applying polycaprolactone to orthodontic research. SEM and EDX data confirmed successful incorporation of calcium and fluoride ions into the polycaprolactone layer, with the amount gradually increasing from the 2.5% to the 5% to the 10% groups. Second, calcium fluoride crystals were applied as the fluoride ion source, which has a low solubility product constant (Ksp=3.9×10⁻¹¹) in the aqueous phase and can gradually release fluoride ions. Using fluoride compounds with high pKa, however, like sodium fluoride (Ksp=9.34×10⁻¹) and tin fluoride, exhibited a high burst release rate in water or saliva mediums (25, 27). Furthermore, calcium fluoride can serve as a fluoride-releasing reservoir system, especially effective at low PH levels (31-33). These features make calcium fluoride a promising candidate for preventing white spot lesions (WSLs). The present invention illustrates the utility of O-rings as carriers to deliver calcium fluoride slowly and consistently, compensating for the low availability of calcium ions in saliva (30).

Moreover, the modified O-rings maintained their elastic strength. No tensile failures were observed in any groups after dip modification. This may be benefited from the similar elasticity modulus of the Ca—F PCL matrix to the O-ring matrix. The 10% group exhibited a significantly increased tensile strength upon stretching, which is likely due to the increased diameter after polycaprolactone layer was coated. In addition, no tensile failures were observed in any groups after 7-week soaking in the distill water, although all the experimental groups had lower final forces than the control group. This indicates that the polycaprolactone layer protect may protect the rubber matrix from swelling or corrosion. While the experimental groups displayed a slight decrease in tensile strength from T1 to T2, these changes were not clinically significant, as all O-rings maintained their structural integrity on the brackets with no apparent shape alteration after 7 weeks of fluoride release. The 5% group, in particular, appears to have the ideal amount of polycaprolactone for incorporating fluoride with long-term therapeutic fluoride release and possessing optimal elastic properties.

The coating method is simple and reliable. Using acetone to dissolve the polycaprolactone and a short dipping time (1 s) for coating largely preserved the matrix of elastomeric ligature tie. Acetone is a mild solvent that has less effect on polyurethane than other polycaprolactone solvents such as dimethyl chloride and chloroform. In addition, the preparation time and protocol are feasible during clinical treatment. Therefore, the present invention not only provides a platform for future basic studies to optimize therapeutic fluoride dose for white spot lesion prevention but also has a clinical translation potential.

In summary, the present invention demonstrates the efficacy of employing polycaprolactone medium and calcium fluoride as innovative approaches to improve sustained fluoride release, reduce burst effects, and maintain the elastic strength of orthodontic O-rings. These findings hold promising implications for orthodontic treatments, offering a potential solution to prevent white spot lesions effectively and efficiently.

The present invention presents an avenue for preventing white spot lesions in orthodontic patients using calcium fluoride O-rings, drawing the following conclusions. The 5% and 10% groups of Ca—F O-rings exhibited sustained and therapeutically effective fluoride release over the long term. The novel dip coating method proved to be efficient and reliable in coating PCL incorporated with calcium fluoride on O-ring surfaces. The Ca—F O-rings maintained their elastic properties after the dip coating modification, ensuring their functional integrity.

The following references are cited herein:
1. Ogaard B. Am J Orthod Dentofacial Orthop. 1989;96(5):423-7.
2. Artun et al., Eur J Orthod. 1986;8(4):229-34.
3. Mizrahi E. Am J Orthod. 1982;82(1):62-7.
4. Gorelick et al., Am J Orthod. 1982;81(2):93-8.
5. Banks et al., Eur J Orthod. 1997;19(4):391-5.
6. Baturina et al., Angle Orthod. 2010;80(6):1129-35.
7. Zimmer et al., J Orofac Orthop. 1999;60(3):205-14.
8. Mellberg et al., J Dent Res. 1984;63(9):1130-5.
9. Jablonowski et al., Quintessence Int. 2012;43(3):221-8.
10. Nascimento et al., Braz Dent J. 2016;27(1):101-7.
11. Azarpazhooh et al., J Am Dent Assoc. 2008;139(7):915-24; quiz 94-5.
12. Perrini et al., Am J Orthod Dentofacial Orthop. 2016;149(2):238-43.
13. Geiger et al., Am J Orthod Dentofacial Orthop. 1988;93(1):29-37.
14. Ogaard et al., Am J Orthod Dentofacial Orthop. 1988;94(2):123-8.
15. Geiger et al., Am J Orthod Dentofacial Orthop. 1992;101(5):403-7.
16. Robertson et al., Am J Orthod Dentofacial Orthop. 2011;140(5):660-8.
17. Pessan et al., J Appl Oral Sci. 2008;16(4):238-46.
18. Marini et al., Eur J Orthod. 1999;21(6):695-701.
19. Wiltshire et al., Am J Orthod Dentofacial Orthop. 1996;110(4):383-7.
20. Wiltshire WA. Am J Orthod Dentofacial Orthop. 1999;115(3):288-92.
21. Storie et al., Angle Orthod. 1994;64(3):199-209; discussion 10.
22. Storie et al., The Angle orthodontist. 1994;64(3):199.
23. Tinsley et al., Journal of Orthodontics. 2003;30(4):317-22.
24. Wiltshire WA. American Journal of Orthodontics & Dentofacial Orthopedics. 1996;110(4):383-7.
25. Banks et al., Eur J Orthod. 2000;22(4):401-7.
26. Doherty et al., Eur J Orthod. 2002;24(4):371-8.
27. Mattick et al., J Orthod. 2001;28(3):217-9.
28. Wilson et al., Am J Orthod Dentofacial Orthop. 1995;107(4):379-81.
29. Tinsley et al., J Orthod. 2003;30(4):317-22; discussion 298.
30. Vogel GL. Monogr Oral Sci. 2011;22:146-57.
31. ten Cate JM. Eur J Oral Sci. 1997;105(5 Pt 2):461-5
32. Rolla G. Acta Odontol Scand. 1988;46(6):341-5.
33. Reynolds et al., J Dent Res. 2008;87(4):344-8.
34. Labet et al., Chem Soc Rev. 2009;38(12):3484-504.
35. Schlesinger et al., Mater Sci Eng C Mater Biol Appl. 2015;57:232-9.
36. Petersen et al., Pharmaceutics. 2020;12(11).
37. Chen et al., Polym Int. 2010;59(8):1154-61.
38. Pawar et al., Polym Advan Technol. 2023;34(10):3296-316.
39. Jing et al., Int J Biol Sci. 2018;14(1):1-9.
40. Jing et al., Sci Rep. 2017;7(1):10020.
41. O'Dwyer et al., Am J Orthod Dentofacial Orthop. 2005;128(4):471-6.
42. Dentistry-Elastomeric auxiliaries for use in orthodontics. 1 ed: International Organization for Standardization; 2007.

What is claimed is:

1. A method for coating an orthodontic elastomeric ligature tie (O-ring) with fluoride, comprising:
preparing a solution of a coating medium and calcium fluoride microcrystals;
coating the O-ring with the solution; and
drying the solution on the O-ring to form a layer of the coating medium with the calcium fluoride microcrystals embedded therein.

2. The method of claim 1, further comprising:
increasing a concentration of the coating medium during the preparing step such that a thicker layer with an increase in an availability of fluoride therein is formed on the O-ring during the coating step.

3. The method of claim 1, wherein the coating medium is polycaprolactone.

4. The method of claim 3, wherein the polycaprolactone is at a concentration of about 2.5% to about 10% in the solution.

5. The method of claim 4, wherein the polycaprolactone is at a concentration of 5% in the solution or 10% in the solution.

6. An orthodontic elastomeric ligature tie (O-ring) prepared by the method of claim 1.

7. The orthodontic elastomeric ligature tie of claim 6 comprising a coating of about 5% to about 10% polycaprolactone with calcium fluoride microcrystals embedded therein.

8. A method for treating demineralization of tooth enamel during an orthodontic treatment in a subject in need thereof, comprising:
attaching the orthodontic elastomeric ligature tie of claim 6 to braces on the teeth, a concentration of the coating medium extending time-release of the fluoride embedded therein as calcium fluoride; and
incorporating the fluoride into the enamel thereby decreasing or preventing demineralization in the tooth enamel.

9. The method of claim 8, wherein incorporating the fluoride into the enamel further increases remineralization of decalcified tooth enamel lesions.

10. The method of claim 8, wherein the coating medium is polycaprolactone, an increasing concentration thereof on the O-ring increasing availability of the fluoride therein to the tooth enamel.

11. The method of claim 10, wherein the concentration of polycaprolactone is about 2.5% to about 10%.

12. The method of claim 11, wherein the concentration of polycaprolactone is 5% or 10%.

13. A method for optimizing time-release of fluoride coated onto an orthodontic elastomeric ligature tie, comprising:
coating the orthodontic elastomeric ligature tie with a polycaprolactone coating medium containing calcium fluoride microcrystals embedded within a matrix thereof, wherein an increase in optimization of the time-release of the fluoride from the polycaprolactone matrix coating the orthodontic elastomeric ligature tie correlates to a concentration of the polycaprolactone thereon.

14. The method of claim 13, wherein the concentration of polycaprolactone is about 5% to about 10%.

15. An oral device to re-mineralize decalcified tooth enamel lesions, comprising:
an orthodontic elastomeric ligature tie; and
a coating medium comprising a polycaprolactone matrix disposed thereon and containing calcium fluoride microcrystals embedded therein.

16. The oral device of claim 15, wherein the polycaprolactone has a concentration of about 2.5% to about 10% within the matrix.

17. The oral device of claim 16, wherein the polycaprolactone has a concentration of about 5% or about 10% within the matrix.

18. The oral device of claim 15, wherein a time-release of fluoride from the polycaprolactone matrix correlates to the concentration of polycaprolactone therein.

19. A method for re-mineralizing decalcified tooth enamel lesions in a subject with orthodontia, comprising:
securing the oral device of claim 15 to the orthodontia in the mouth of the subject; and
time-releasing fluoride from the calcium fluoride embedded within the polycaprolactone, said time-releasing dependent on the concentration of the polycaprolactone within the polycaprolactone; wherein the concentration is about 2.5% to about 10%.

20. A kit for modifying an orthodontic elastomeric ligature tie to release fluoride as a therapeutic, comprising:
polycaprolactone;
calcium fluoride;
at least one solvent to prepare a coating medium of the polycaprolactone and calcium fluoride; and
instructions to prepare calcium fluoride coated O-rings.
